(19) United States
(12) Patent Application Publication
(10) Pub. No.: US 2022/0192831 A1
(43) Pub. Date: Jun. 23, 2022

(54) ADAPTABLE DEVICES AND SYSTEMS FOR DOCKING IN CIRCULATORY SYSTEM AND METHODS THEREOF

(71) Applicant: Edwards Lifesciences Corporation, Irvine, CA (US)

(72) Inventors: Nikolay Gurovich, Hadera (IL); Michael Bukin, Pardes Hanna (IL); Boaz Manash, Givat Ada (IL); Eason Michael Abbott, Santa Monica, CA (US); John J. Desrosiers, San Clemente, CA (US); Christine Marie Heyninck-Jantz, Trabuco Canyon, CA (US)

(21) Appl. No.: 17/654,570

(22) Filed: Mar. 11, 2022

Related U.S. Application Data
(63) Continuation of application No. PCT/US2020/050059, filed on Sep. 10, 2020.
(60) Provisional application No. 62/900,059, filed on Sep. 13, 2019.

(51) Int. Cl. A61F 2/24; A61F 2/00

(52) U.S. Cl. CPC A61F 2/2475; A61F 2/0077; A61F 2250/001; A61F 2230/006; A61F 2220/0016; A61F 2230/0054

(57) ABSTRACT

Devices, systems and methods for situating a device within a variety vasculature morphologies are provided. An expandable device can adapt to the local vasculature morphology and still provide a controlled inner diameter is described. A device for situating within the inferior vena cava and superior vena cava is described. Systems and methods for utilizing devices are described.

### CROSS-REFERENCE TO RELATED APPLICATIONS

This application is a continuation of International Application No. PCT/US2020/050059 filed Sep. 10, 2020, and entitled “ADAPTABLE DEVICES AND SYSTEMS FOR DOCKING IN CIRCULATORY SYSTEM AND METHODS THEREOF,” which claims the benefit of priority to U.S. Prov. App. No. 62/900,059 filed Sep. 13, 2019, the entirety of each of these applications being incorporated by reference herein for all purposes.

### FIELD OF THE INVENTION

The application is generally directed to devices and systems to dock within the circulatory system, and more specifically to devices and systems that adapt shape to the local environment.

### BACKGROUND

A number of prosthetics can be utilized to treat a variety of cardiac and circulatory disorders. For instance, prosthetic heart valves can be utilized to treat valvular disorders such as valvular insufficiency. Likewise, prosthetics may be useful in repair or bypass of an aneurysm.

A transcatheter technique for introducing and implanting a prosthetic using a catheter in a manner that is less invasive and can reduce complications associated with various surgical procedures (e.g., open heart surgery). In this technique, a prosthetic can be mounted in a crimped state on the end portion of a catheter and advanced through a blood vessel of the patient until the prosthetic reaches the implantation site. The prosthetic at the catheter tip can then be expanded to its functional size at the site of repair, such as by inflating a balloon or utilizing self-expanding stent or frame. Optionally, a prosthetic can have a balloon-expandable, self-expanding, mechanically-expandable frame, and/or a frame expandable in multiple or a combination of ways. One common prosthetic utilized in a transcatheter technique are heart valves (THV).

### SUMMARY OF THE INVENTION

Many embodiments are directed to towards devices that can implant within the vasculature and adapt to the shape of the local environment.

In an embodiment of an expandable device for implanting within the vasculature, an expandable device includes a central core that includes a number of struts that form a number of cells, the cells forming a circumference encircling the central core. The central core is capable of expanding from an unexpanded state radially outward to an expanded state. When the central core is an expanded state, the central core has a controlled inner diameter. The expandable device includes a plurality of appendages that extend away from the central core, each appendage is formed by at least one strut and is independent from the other appendages. Each appendage is capable of independently expanding from an unexpanded state radially outward to an expanded state such that the apex of each appendage extends radially outward beyond the outer circumference of the central core.

In another embodiment, the controlled inner diameter of the central core in an expanded state is between 15 and 30 mm.

In yet another embodiment, the controlled inner diameter of the central core in an expanded state is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In a further embodiment, the length of the central core is longer in an unexpanded state as compared to an expanded state.

In still yet another embodiment, the width or thickness of at least one strut varies.

In yet a further embodiment, at least one appendage is formed by a single strut.

In an even further embodiment, at least one appendage is formed by at least two struts that connect at the apex of the appendage.

In yet an even further embodiment at least one appendage forms a V-shape.

In still yet an even further embodiment, at least one appendage forms a Y-shape.

In still yet an even further embodiment, at least one appendage has an orifice at the apex.

In still yet an even further embodiment, at least one appendage has a hook at the apex.

In still yet an even further embodiment, the expandable device further includes a radiopaque portion within the central core or at least one appendage.

In still yet an even further embodiment, the central core and appendages are made of a high radial strength material.

In still yet an even further embodiment, the high radial strength material is cobalt chromium or stainless steel.

In still yet an even further embodiment, the central core and appendages are made of a self-expanding material.

In still yet an even further embodiment, the self-expanding material is nitinol.

In still yet an even further embodiment, a cover is incorporated within the central core.

In still yet an even further embodiment, the cover is secured to the struts of the central core.

In still yet an even further embodiment, the cover is impermeable to blood.

In still yet an even further embodiment, the cover is a biocompatible fabric.

In still yet an even further embodiment, the cover is a bioprosthetic tissue.

In still yet an even further embodiment, the cover is further incorporated within the plurality of appendages and includes sealing portions that are capable of engaging an inner wall at a site of deployment.

In still yet an even further embodiment, the central core is configured to engage a prosthesis.

In still yet an even further embodiment, the central core includes a valve seat configured to situate a prosthetic valve.

In still yet an even further embodiment, the outer perimeter of the prosthetic valve is compatible with the inner perimeter of the central core.

In still yet an even further embodiment, the outer diameter of the prosthetic valve is between 15 mm and 30 mm.

In still yet an even further embodiment, the outer diameter of the prosthetic valve is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In still yet an even further embodiment, when the central core and the plurality of appendages are in a crimped state, the central core and the plurality of appendages are configured to be inserted within a catheter.

In still yet an even further embodiment, when in an expanded state, the apex of each appendage is capable of engaging an inner wall at a site of deployment.

In still yet an even further embodiment, the inner wall is a luminal wall of a vasculature.

In still yet an even further embodiment, the vasculature at the site of deployment is dilated.

In still yet an even further embodiment, the vasculature at the site of deployment is experiencing an aneurysm.

In an embodiment of a method of implanting an expandable device, an expandable device is delivered to a site of deployment. The expandable device includes a central core and a plurality of appendages that extend away from the central core. The central core comprises a number of struts that form a number of cells, the cells forming a circumference encircling the central core. Each appendage is formed by at least one strut and is independent from the other appendages. The expandable device is delivered in an unexpanded state. The expandable device is expanded from the unexpanded state into an expanded state such that the central core is expanded to a controlled inner diameter and each apex of each appendage of the plurality of appendages expands beyond the outer circumference of the central core.

In another embodiment, the controlled inner diameter of the central core in an expanded state is between 15 and 30 mm.

In yet another embodiment, the controlled inner diameter of the central core in an expanded state is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In a further embodiment, the length of the central core is longer in an unexpanded state as compared to an expanded state.

In still yet another embodiment, the expansion of the expandable device is monitored utilizing radiopaque portions of the expandable device.

In yet a further embodiment, the expandable device is expanded by an inflatable balloon.

In an even further embodiment, the expandable device is expanded mechanically.

In yet an even further embodiment, the expandable device is self-expandable.

In still yet an even further embodiment, the site of deployment is within mammalian vasculature.

In still yet an even further embodiment, the site of deployment is within an anthropomorphic phantom.

In various embodiments, the methods can be performed on a living animal or on a non-living cadaver, cadaver heart, simulator (e.g. with the body parts, tissue, etc. being simulated), anthropomorphic phantom, etc.

In still yet an even further embodiment, the inner wall at the site of deployment is engaged with at least one appendage of the plurality of appendages.

In still yet an even further embodiment, a prosthetic valve is expanded within the inner circumference of the central core such that outer of the prosthetic valve engages with a valve seat within the central core.

In an embodiment of a docking station for implanting within the vasculature, a docking station incorporating a central section that includes a number of struts that form a number of cells, the cells forming a circumference encircling the central section. The central section is capable of expanding from an unexpanded state radially outward to an expanded state. When the central section is an expanded state, the central section includes a valve seat with an inner diameter. The docking station includes an inflow section. The docking station includes an outflow section incorporating an arm that is bent outwardly into an elbow-like conformation. The central section is connected to and in between the inflow and the outflow sections. The inflow and outflow sections are capable of expanding from an unexpanded state radially outward to an expanded state. The outer circumferences of the inflow and outflow sections are each greater than the outer circumference of the central section.

In another embodiment, the inner diameter of the valve seat in an expanded state is between 15 and 30 mm.

In yet another embodiment, the inner diameter of the valve seat in an expanded state is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In a further embodiment, the length of the docking station is longer in an unexpanded state as compared to an expanded state.

In still yet another embodiment, the width or thickness of at least one strut varies.

In yet a further embodiment, the arm is a cell formed by a number of struts.

In an even further embodiment, the outflow section includes a number of struts that form a number of cells, the cells forming a circumference partially encircling the outflow section such that a gap is formed in the outflow section.

In yet an even further embodiment, the arm is situated within the gap.

In still yet an even further embodiment, the inflow section includes a number of struts that form a number of cells, the cells forming a circumference encircling the inflow section.

In still yet an even further embodiment, the docking station includes a radiopaque portion connected to the docking station.

In still yet an even further embodiment, the at least one radiopaque portion is integrated such that the bent arm is a capable of being identified using radiography.

In still yet an even further embodiment, the docking station is made of a self-expanding material.

In still yet an even further embodiment, the self-expanding material is nitinol.

In still yet an even further embodiment, a cover is incorporated within the docking station.

In still yet an even further embodiment, the cover is secured to the struts of the central section and to inflow section via sutures.

In still yet an even further embodiment, the cover is impermeable to blood.

In still yet an even further embodiment, the cover is a biocompatible fabric.

In still yet an even further embodiment, the cover is a bioprosthetic tissue.

In still yet an even further embodiment, sealing portions are formed onto the cover that are capable of engaging an inner wall at a site of deployment.

In still yet an even further embodiment, the valve seat is configured to situate a prosthetic valve.

In still yet an even further embodiment, the outer perimeter of the prosthetic valve is compatible with the inner perimeter of the valve seat.

In still yet an even further embodiment, the outer diameter of the prosthetic valve is between 15 mm and 30 mm.

In still yet an even further embodiment, the outer diameter of the prosthetic valve is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In still yet an even further embodiment, when the docking station is in a crimped state, the docking station is configured to be inserted within a catheter.

In still yet an even further embodiment, when in an expanded state, the inflow and outflow sections are capable of engaging an inner wall at a site of deployment.

In still yet an even further embodiment, the inner wall is a luminal wall of a vasculature.

In still yet an even further embodiment, the vasculature at the site of deployment is the inferior vena cava or the superior vena cava.

In still yet an even further embodiment, the arm is configured to engage a wall within the right atrium.

In an embodiment of a method of implanting a docking station, a docking station is delivered to a site of deployment, the docking station includes a central section, an inflow section, and outflow section, and an arm. The central section includes a number of struts that form a number of cells, the cells forming a circumference encircling the central section. The inflow section includes a number of struts that form a number of cells, the cells forming a circumference encircling the inflow section. The outflow section includes a number of struts that form a number of cells, the cells forming a circumference partially encircling the outflow section such a gap is formed in the outflow section. The arm is situated within the gap and is capable of bending into an elbow-like shape. The central section is connected to and in between the inflow and the outflow sections. The expandable device is delivered in an unexpanded state. The docking station is expanded from the unexpanded state into an expanded state such that the central section is expanded to form a valve seat having an inner diameter and such that outer circumferences of the inflow and outflow sections are each greater than the outer circumference of the central section.

In another embodiment, the inner diameter of the valve seat in an expanded state is between 15 and 30 mm.

In yet another embodiment, the inner diameter of the valve seat in an expanded state is: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm or 30 mm.

In a further embodiment, the length of the docking station is longer in an unexpanded state as compared to an expanded state.

In still yet another embodiment, the expansion of the docking station is monitored utilizing radiopaque portions of the expandable device.

In yet a further embodiment, the expandable device is self-expandable.

In an even further embodiment, the site of deployment is within mammalian vasculature.

In yet an even further embodiment, the site of deployment is the inferior vena cava or the superior vena cava.

In still yet an even further embodiment, an inner luminal wall at the site of deployment is engaged with the inflow section, and the right atrium wall is engaged with the arm.

In still yet an even further embodiment, the site of deployment is within an anthropomorphic phantom.

In various embodiments, the methods can be performed on a living animal or on a non-living cadaver, cadaver heart, simulator (e.g. with the body parts, tissue, etc. being simulated), anthropomorphic phantom, etc.

In still yet an even further embodiment, an inner wall at the site of deployment is engaged with inflow section.

In still yet an even further embodiment, a prosthetic valve is expanded within the valve seat such that an outer wall of the prosthetic valve engages with the valve seat.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an illustration of the human heart.

FIG. 2A to FIG. 2E each provide an illustration of morphologies of vasculature, including straight, slight dilation, curvy, fusiform aneurysm, and saccular aneurysm.

FIG. 3A provides a perspective view illustration of an expandable device with independent appendages in accordance with an embodiment.

FIG. 3B provides a perspective view illustration of an expandable device with multiple rows of cells in the central core in accordance with an embodiment.

FIG. 3C provides a perspective view illustration of an expandable device with single strut appendages in accordance with an embodiment.

FIG. 3D provides a zoomed-in illustration of orifices at the apex of appendages in accordance with an embodiment.

FIG. 3E provides a zoomed-in illustration of hooks at the apex of appendages in accordance with an embodiment.

FIG. 4 provides a perspective view illustration of an expandable device in unexpanded form in accordance with an embodiment.

FIG. 5A provides a perspective view illustration of an expandable device with a full length cover in accordance with an embodiment.

FIG. 5B provides a perspective view illustration of an expandable device with a partial length cover in accordance with an embodiment.

FIG. 6 provides a perspective view illustration of an expandable device with a prosthetic valve in accordance with an embodiment.

FIG. 7A to FIG. 7E each provide an illustration of an expandable device in various morphologies of vasculature, including straight, slight dilation, curvy, fusiform aneurysm and saccular aneurysm, in accordance with various embodiments of the invention.

FIG. 8A provides a side view illustration of a docking station with bent arm in accordance with an embodiment.

FIG. 8B provides a side view illustration of a docking station with bent arm in accordance with an embodiment.

FIG. 9 provides a side view illustration of a docking station with bent arm in unexpanded form in accordance with an embodiment.

FIG. 10A provides a perspective view illustration of a docking station with bent arm with a full length cover in accordance with an embodiment.

FIG. 10B provides a perspective view illustration of a docking station with bent arm with a partial length cover in accordance with an embodiment.

FIG. 11 provides a perspective view illustration of a docking station with bent arm with a prosthetic valve in accordance with an embodiment.

FIGS. 12A to 12C provide illustrations of a docking station with bent arm situated within the inferior vena cava in accordance with various embodiments.

### DETAILED DESCRIPTION

Turning now to the drawings, devices and systems for docking within the circulatory system are described, in accordance with various embodiments of the invention. In many embodiments, devices and methods are described for providing a docking station within a vein, artery, valvular space, or other component of the circulatory system. In several embodiments, a docking station is configured to dock a prosthetic (e.g., prosthetic heart valve).

The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Various embodiments of docking stations/devices and examples of prosthetic valves or transcatheter valves are disclosed herein, and any combination of these options can be made unless specifically excluded. For example, any of the docking stations/devices disclosed, can be used with any type of valve, and/or any delivery system, even if a specific combination is not explicitly described. Likewise, the different constructions and features of docking stations/devices and valves can be mixed and matched, such as by combining any docking station type/feature, valve type/feature, tissue cover, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

### Overview of Heart and Circulatory Anatomy

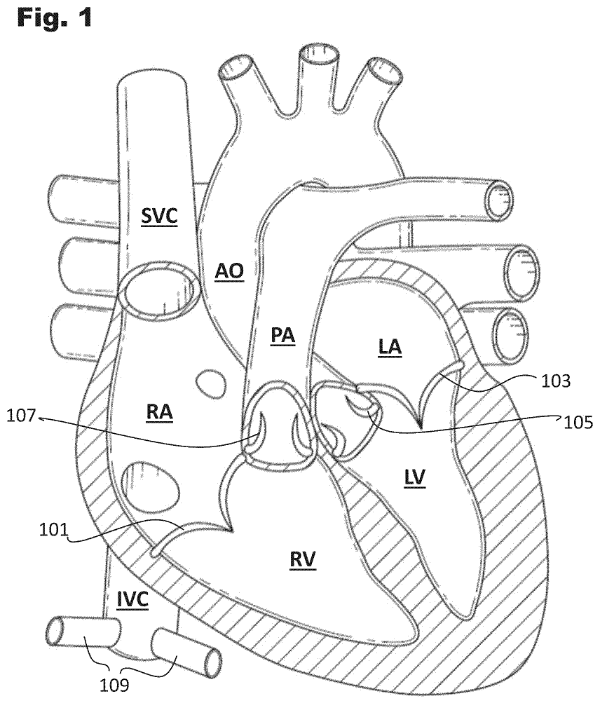

FIG. 1 is a cutaway view of the human heart in a diastolic phase. The right ventricle (RV) and left ventricle (LV) are separated from the right atrium (RA) and left atrium (LA), respectively, by the tricuspid valve 101 and mitral valve 103; i.e., the atrioventricular valves. Additionally, the aortic valve 105 separates the LV from the ascending aorta (AO) and the pulmonary valve 107 separates the RV from the pulmonary artery (PA). Each of these valves has flexible leaflets extending inward across the respective orifices that come together or “coapt” in the flowstream to form the one-way, fluid-occluding surfaces. The docking stations and valves of the present application are described, for illustration, may be utilized within the inferior vena cava (IVC), superior vena cava (SVC), aorta/aortic valve, or other location. A defective aortic valve, for example, can be a stenotic aortic valve and/or suffer from insufficiency and/or regurgitation. The blood vessels, such as the aorta, IVC, SVC, pulmonary artery, may be healthy or may be dilated, distorted, enlarged, have an aneurysm, or be otherwise impaired. Anatomical structures of RA, RV, LA, and LV will be explained in greater detail. The devices described herein can be used in various areas whether explicitly described herein or not, e.g., in the IVC and/or SVC, in the aorta, and other locations as treatment for a defective valve, areas of aneurysm, and other circulatory disorders.

The RA receives deoxygenated blood from the venous system through the SVC and the IVC, the former entering the RA from above, and the latter from below. During the diastolic phase, or diastole, seen in FIG. 1, the deoxygenated blood from the IVC, and SVC that has collected in the right atrium RA passes through the TV and into the RV as the RV expands. In the systolic phase, or systole, the right ventricle RV contracts to force the deoxygenated blood collected in the RV through the pulmonary valve PV and pulmonary artery into the lungs. Also shown are the hepatic veins 109, which transfer deoxygenated blood from the liver to the IVC.

Adaptable Devices with Controlled Inner Diameter

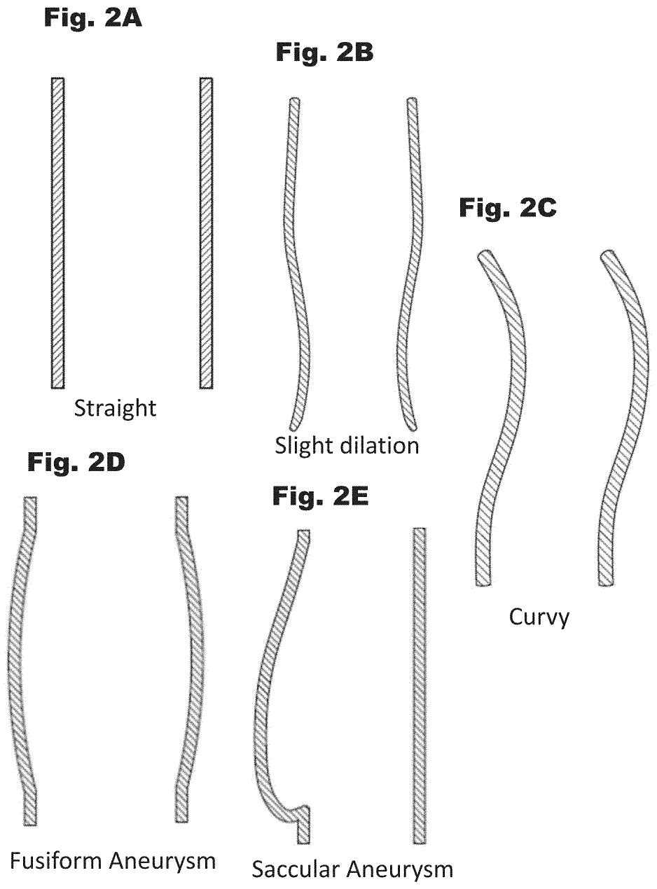

Referring to FIGS. 2A-2E, non-exhaustive examples illustrate that the vasculature can have a wide variety of different shapes and sizes. For example, as shown in FIGS. 2A-2E, the length, diameter, and curvature or contour may vary greatly between vasculatures of different patients, especially within the IVC, SVC, and aorta. These differences can be even more significant in vasculatures that suffer from certain conditions such as an aneurysm. For example, a fusiform aneurysm (depicted in FIG. 2D) and a saccular aneurysm (depicted in FIG. 2E) can result in oddly shaped and dilated vascular walls, often resulting in lack of symmetry.

Accordingly, implanting docking stations and/or devices having a fixed shape and diameter within the vasculature can be difficult due to individual variations. To overcome issues of varied vascular morphologies, devices are described herein that can adjust to the local vascular dimensions.

In accordance with several embodiments, an expandable device capable of adapting to the size, contour and dimensionality of the local environment within the vasculature while maintaining a controlled inner diameter is described. In many embodiments, an expandable device is a cylindrical frame and has central core in the middle portion and two sections extending distally away from the central core along the length of the cylindrical frame. In numerous embodiments, when expanded, a central core has a controlled inner diameter. In multiple embodiments, each of the two distal sections have a number of appendages extending off of and away from the central core and along the length of the cylindrical frame. In accordance with many embodiments, an expandable device can expand radially outward from an unexpanded form such that the central core expands radially outward to a controlled interior diameter and the appendages each independently expand radially outward such that the apex is beyond the outer circumference of the central core. In some embodiments, a number of appendages independently expand radially outward to an inner luminal wall at the site of deployment (e.g., within the vasculature) such that the expandable devices fit to the local dimensionality of the deployment site and maintains a controlled inner diameter.

In several embodiments, a central core is composed of a number of interconnecting struts to form a number of cells. In numerous embodiments, each cell within the central core is connected and/or adjacent to another cell to form a row of cells, the row of cells encircles the circumference of the central core. In some embodiments, the central core includes multiple rows of cells, each row circulating around the circumference of the central core. It should be understood that the perimeter of a cell can vary, depending on the length of the struts that form the cell. It should be further understood that the thickness and/or width of a strut can vary, which may be beneficial to provide rigidity or malleability along some portions of the strut.

In a number of embodiments, an expandable device has an unexpanded conformation, typically with an elongated length and a much smaller circumference, as compared to an expanded conformation. In various embodiments, when an expandable device is expanded outwardly, the device length is shortened and the device circumference is increased. In other words, in various embodiments, the length of the central core is longer in an unexpanded state as compared to an expanded state. Accordingly, in several embodiments, an expandable device can be expanded at the site of deployment, such that it is expanded to reach outward to the inner walls of a location, such as the luminal walls of the vasculature. In many embodiments, a central core has an expanded circumference that is restricted such that the internal diameter of the central core is controlled regardless of the lumen shape and circumference at the site of deployment. Controlling the diameter size may provide benefits, especially when a particular diameter is useful for various applications, such as (for example) when the expandable device is used as a docking station for a prosthetic or when used to advance blood flow through dilated areas (e.g., aneurysm).

In many embodiments, an expandable device has a central core with a controlled internal diameter, the internal diameter is between 15 mm and 35 mm. In various embodiments, an internal diameter is approximately one of: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm. It is to be understood, however, that the controlled inner diameter can be any appropriate size to house a prosthetic or to promote blood flow through dilated areas.

In various embodiments, a number of appendages of the expandable device extend distally on one or both ends along the length from the central core. Accordingly, the most distal points of the appendages formulate the upper end and/or the lower end, each end having a circumference and diameter. It is noted, for the sake of ease of description, when an expandable device is inserted within the vasculature, a lower end confers the inflow end and the upper end confers the outflow end in accordance with flow of blood. In some embodiments, an upper and/or a lower diameter is larger than the diameter of a central core, conferring a “dog bone” shape. In other words, in some embodiments, the apex of an appendage is capable of expanding in an outward direction beyond the circumference of the central core, which may be useful to reach inner walls in areas of deployment, such as luminal walls of the vasculature where the vasculature is dilated and/or aneurytic. In many embodiments, a number of appendages of the expandable device are isolated from the other appendages such that the isolated appendages can solitarily expand outward. In some embodiments, each appendage of the expandable device is isolated from the other appendages such that each appendage can solitarily expand outward. Accordingly, an upper and lower circumference may not be a circle, but would depend on how far each appendage expands outward. Appendages, in accordance with various embodiments, can be formulated by a single strut, multiple struts (e.g., two struts extending to the distal apex to form a V-shape), or any combination thereof (e.g., two struts reaching to a connecting point and a third strut expanding outward to form a Y-shape). It should be understood that the length and width of an appendage can vary, depending on the dimensions of the struts used to formulate the appendage. It should be further understood that the thickness and/or width of a strut can vary, which may be beneficial to provide rigidity or malleability along some portions of the strut.

In a number embodiments, appendages reach out distally to a diameter of at least 40 mm to at least 55 mm. In some embodiments, appendages reach out distally to a diameter of at least 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, or 55 mm. It is to be understood, however, that the reach of the distal appendages can be any appropriate size to fit within and reach to the inner luminal walls at the site of implantation. Further, it is to be understood that the reach the reach of the distal appendages can be non-uniform meaning that some appendages can reach out to a further diameter than other appendages.

In many embodiments, appendages have a means for securing an expandable device's position and location at the site of deployment, which may mitigate an expanded and implanted device from sliding and/or dislodging from the deployment site. In several embodiments, at least some appendages have an orifice near the apex, which may be useful for suturing. In some embodiments, threading orifices at the distal end of appendages allows one to control the expansion of the appendages, which may help control the diameter of the appendages. In some embodiments, an expandable device is sutured at the site of deployment. In a number of embodiments, at least some appendages have a hook near the distal end, which may be useful for gripping onto an inner wall at the site of deployment. In some embodiments, an appendage would have an orifice and a hook near the distal end. Although orifices and hooks are described as means for securing appendages to the site of deployment, it should be understood that other means, as understood in the art, could also be used and fall within some embodiments of the invention.

In some embodiments, appendages may be shaped and/or have radiopaque qualities at various points along the appendage, including the distal apex, such that the appendage can be easily recognized utilizing appropriate radiographic imaging technique (e.g., sonography). Facilitating visualization of the appendages can be useful to ensure that the appendages expand outwardly as desired by the user.

In several embodiments, an expandable device is composed of a resilient and compliant material capable of expanding at the site of deployment. In some embodiments, an expandable device is made of a high radial strength material such as (for example) cobalt chromium or stainless steel. When a high radial strength material is utilized, it can help control the internal diameter of a central core of the expandable device. In some embodiments, an expandable device is made of a self-expanding material such as (for example) nitinol. A self-expanding material may continue to expand within the site of deployment, helping it to secure within its place by radial expansion forces. In some embodiments, an expandable device is composed of multiple materials. For example, in some embodiments, the central core of an expandable device is made of a resilient and compliant material (e.g., cobalt chromium or stainless steel) and the appendages are made of a self-expanding material (e.g., nitinol).

In a number of embodiments, a skirt cover is attached to the expandable device such that a wall is formed around the outer circumference and along the length of the device and a lumen is formed within the cover, having an opening at both ends of the expandable device. In many embodiments, a cover is attached and secured at the ends of at the expandable device such that the cover extends from end to end. In some embodiments, a cover is also attached along the circumference of a central core. In some embodiments, a cover is only attached to a portion of the expandable device, such that a wall is formed for only a portion along the length of the expandable device. In some such embodiments, a partial cover covers the inflow end and/or the outflow end and/or the central core. In some embodiment, a partial cover covers the inflow end and central core, leaving the outflow end relatively open. In some embodiments, a cover is attached to the inner wall of the expandable device. In some embodiments, a cover is attached to the outer wall of the expandable device. In some embodiments, a cover is attached to both the inner wall and outer wall of the expandable device.

In many embodiments, a cover is impermeable or semi-permeable to blood and constituents of the circulatory system. In several embodiments, a cover is a biocompatible fabric (e.g., PET cloth) or a bioprosthetic tissue. Examples of bioprosthetic tissue include (but are not limited to) animal pericardium and small intestinal submucosa (SIS). Bioprosthetic tissue can be derived from any appropriate animal source, including (but not limited to) bovine, porcine, ovine, avian, and human donor.

In various embodiments utilizing an impermeable cover, sealing portions of a cover can be utilized to seal an expandable device to an inner wall, which, when situated within the vasculature, ensures blood flow through the device and not around it. In several embodiments, a sealing portion is integrally formed onto the body of an expandable device with a cover, in which a portion of the cover makes contact with the luminal wall when expanded at the site of deployment. In many embodiments, a sealing portion is utilized on the inflow end of a device. In some embodiments, a sealing portion is utilized on the outflow end of a device. Utilizing a cover with sealing portions on an expandable device can be beneficial to bypass dilated areas of the vasculature and/or getting blood to flow through the central core, which may situate a prosthetic device such as a prosthetic valve.

In several embodiments, an expandable device can be utilized as a docking station to situate a prosthetic at a certain location within the site of deployment. In many embodiments, a prosthetic valve is utilized with an expandable device, helping control the flow of blood and mitigating backflow, especially in areas of the vasculature that are dilated. In multiple embodiments, the central core of an expandable device is utilized as a valve seat, such that a valve can situate in the central core. In some embodiments, the controlled inner diameter of an expandable device's central core is compatible with the outer diameter of the prosthetic. Having a controlled inner diameter can help ensure that a manufactured prosthetic valve having a standard diameter will work in any local vascular environment, despite the size, contour and dimensionality of the local environment.

In many embodiments, a prosthetic device has an outer diameter, the outer diameter is between 15 mm and 35 mm. In various embodiments, an outer diameter is approximately one of: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm. It is to be understood, however, that the prosthetic device outer diameter can be any appropriate size and a docking station inner diameter can be utilized to be compatible the outer diameter of the prosthetic device.

In some embodiments, a prosthetic device is self-expanding, utilizing radial forces to secure itself within the central core of a docking station. In some embodiments, a mechanism is utilized to secure a prosthetic device with the central core of a docking station, such as (for example) a latch or hook.

A number of prosthetic valves can be utilized with an expanding device, including (but not limited to) mechanical and tissue-based valves. A number of valves have been described and can be utilized, including valves described in U.S. Pat. No. 8,002,825, Published Patent Cooperation Treaty Application No. WO 2000/42950, U.S. Pat. Nos. 5,928,281, 6,558,418, 6,540,782, 3,365,728, 3,824,629, and 5,814,099, the disclosures of which are each incorporated herein by reference in each patent's or publication's entirety. In some embodiments, the Edwards Lifescience's SAPIEN Transcatheter Heart Valve is utilized. It is noted that any appropriate valve capable of fitting within an expandable device, as described herein, can be utilized in accordance with various embodiments of the invention.

In many embodiments, an expandable device can be inserted within a transcatheter delivery device. Accordingly, an expandable device in a crimped stated (e.g., unexpanded form) is inserted within a catheter such that the expandable device can be delivered via the transcatheter approach to a site of implementation. Any appropriate transcatheter delivery system can be employed. In some embodiments, an expandable device utilizes a transcatheter delivery system described in the US Patent Publication No. 2017/0231756, the disclosure of which is incorporated herein by reference it its entirety.

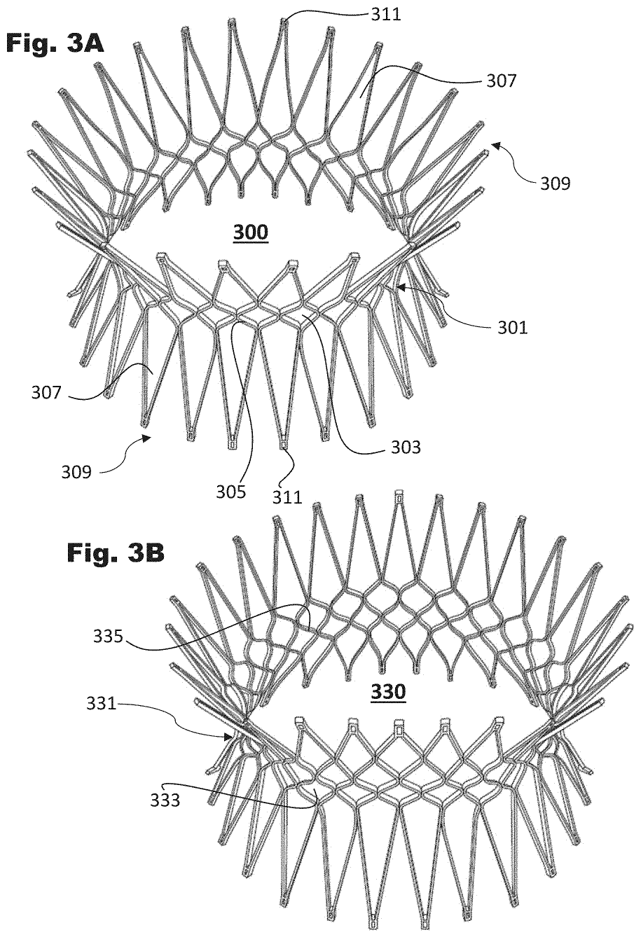

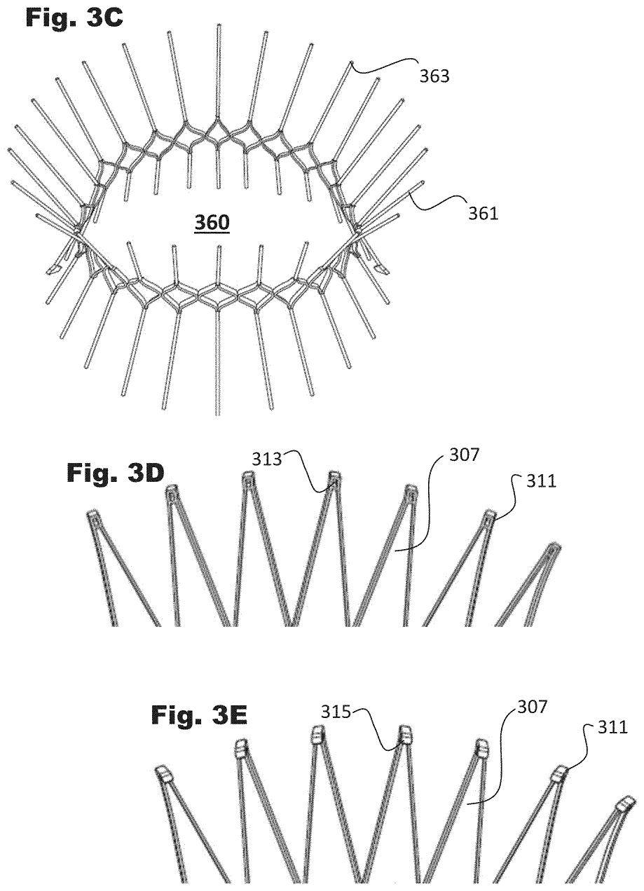

Provided in FIGS. 3A to 3E are various embodiments of an expandable device adapting to the size, contour and dimensionality of the local environment within the vasculature while maintaining a controlled inner diameter. As shown in FIG. 3A, expandable device 300, is presented in its expanded form. Expandable device 300 has a central core 301 having a number of cells 303 formed by a plurality of struts 305. The cells 303 within the central core 301 are adjacent to one another such that a row of cells that encircle the circumference of the central core. Extending from the central core 301 are a number of appendages 307 that are each independent from the other appendages, which can allow each appendage to expand outwardly on its own. Notably, the diameter 309 formed at the distal apices 311 of the appendages 307 in both directions is greater than the inner diameter of the central core 301. The inner diameter of the central core 301 is expanded to a controlled length.

As shown in FIG. 3B, expandable device 330 has a central core 331 having a number of cells 333 formed a plurality of struts 335. In this embodiment, the central core 331 has two rows of cells 333 that encircle the circumference of the central core.

Returning back to FIG. 3A, the appendages 307 are each formed by two struts that join at a distal apex 311. As shown in FIG. 3C, expandable device 360 includes appendages 361 that are each formed by a single strut that each extend out to a distal apex 363. FIG. 3D shows distal apices 311 having an orifice 313. FIG. 3E shows distal apices 311 having a hook 315.

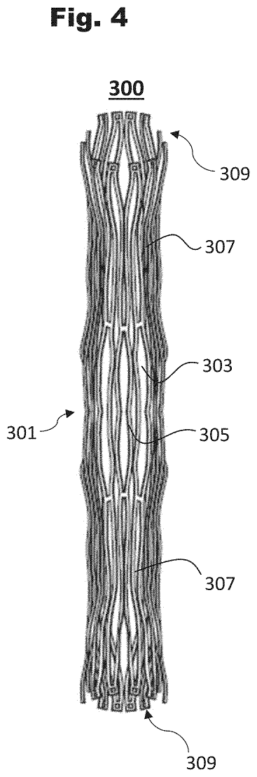

Provided in FIG. 4 is an expandable device 300 in an unexpanded configuration and/or crimped state. As shown the cylindrical diameter is reduced while the length of the device is expanded. The expandable device 300 in a crimped state can be inserted within a catheter such that it can be delivered by a transcatheter approach.

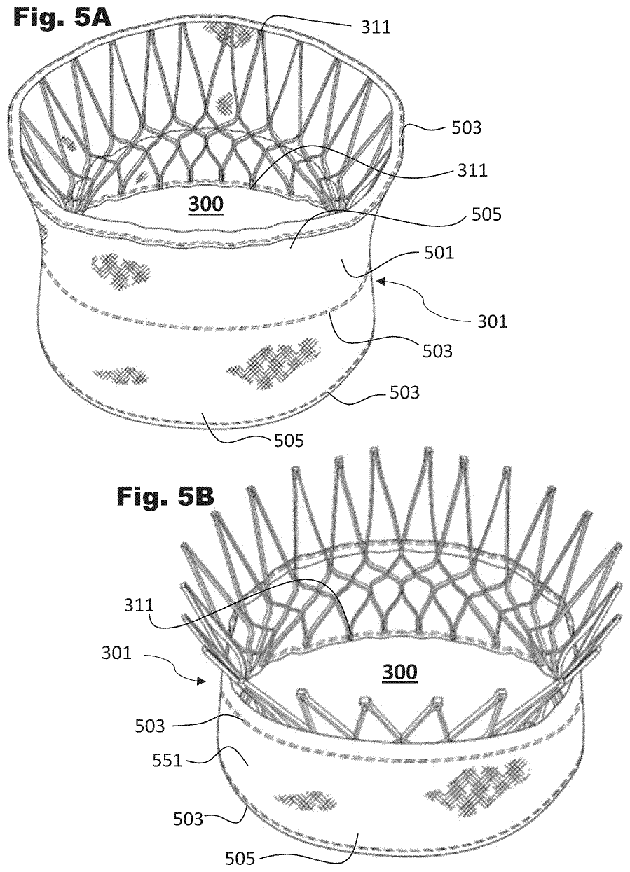

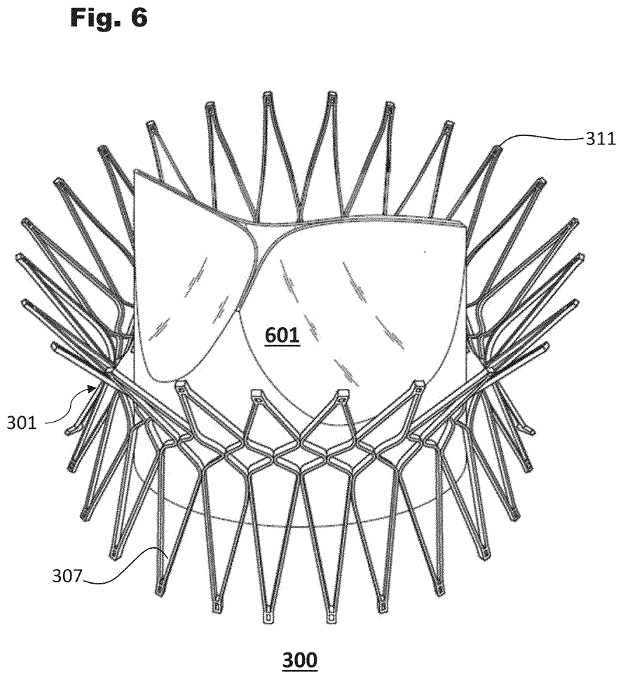

FIGS. 5A and 5B portray expandable device 300 with a cover attached thereupon. A full length cover 501 that extends from a distal apex 311 to the opposite distal apex 311 is shown in FIG. 5A. Alternatively a partial length cover 551 that extends from distal apex 311 to just beyond the central core 301 is shown in FIG. 5B. The full length cover 501 and partial length cover 551 can be attached to the frame apices 311 and/or to the perimeter of central core 301 via stitching 503 or any other appropriate means. The covers 501 and 551 can be made of an appropriate material, such as (for example) a biocompatible fabric (e.g., PET cloth) or a bioprosthetic tissue (e.g., animal pericardium or small intestinal submucosa). Furthermore, the covers 501 and 551 can be made impermeable to blood and its constituents such that it can guide the blood flow throw the central core 301.

Sealing portions 505 are formed on the covers 501 and 551 towards the distal ends. The sealing portions 505 can engage with an inner wall at the site of deployment. Having sealing portions 505 engaged with a luminal wall forms a seal that ensures that blood flows through the central core 301 and does not bypass.

Provided in FIG. 6 is expandable device 300 utilized as a docking station to support a prosthetic valve 601. The valve 601 is situated within the central core 301. The inner circumference of the central core 301 can be controlled such that it is compatible with the outer circumference of the prosthetic valve 601. The prosthetic valve 601 can be self-expanding and utilize radial forces to secure itself within the central core 301 or a mechanism can be utilized to secure the prosthetic valve 601, such as (for example) a latch or hook. Any appropriate prosthetic valve 601 may be utilized, including (but not limited to) mechanical and tissue-based valves. It is noted that the prosthetic valve 601 can be incorporated within expandable device 300 having a cover 501 or 551, which would help guide the blood flow through the prosthetic valve.

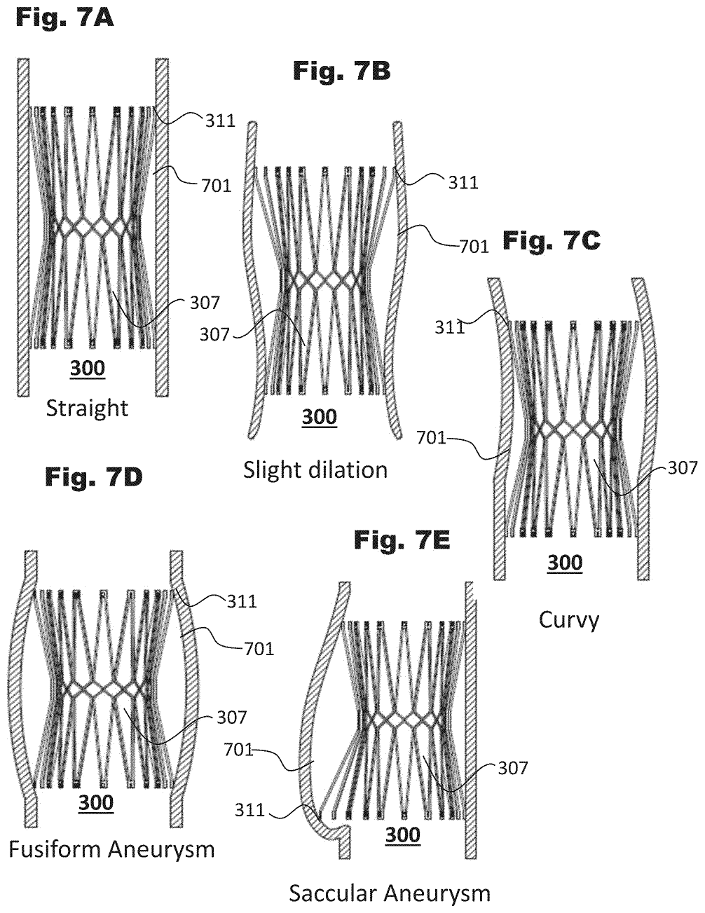

FIGS. 7A to 7E portray expandable frame 300 inserted with various vasculatures. When inserted at the site of deployment, the appendages 307 expand outward and engage with the luminal wall 701. Each appendage 307 can expand outwardly on its own, which allows for engage with the luminal wall 701 even when local vasculature has an irregular shape, including (but not limited to) dilated, curvy, fusiform aneurytic and saccular aneurytic conformations. The appendages can be secured to luminal wall utilizing radial force and/or an attachment mechanism. Attachment mechanisms include (but are not limited to) utilizes orifices 313 and sutures or hooks 315. Despite insertion within various vasculature formations, the central core 301 can maintain a controlled inner diameter. Although not shown in FIGS. 7A to 7E, a prosthetic valve 601 and a cover 501 or 551 can also be utilized.

A number of embodiments are directed to methods of delivering an expandable device to the site of deployment. A method can be performed on any suitable recipient, including (but not limited to) humans, other mammals (e.g., porcine), cadavers, or anthropomorphic phantoms, as would be understood in the art. Accordingly, methods of delivery include both methods of treatment (e.g., treatment of human subjects) and methods of training and/or practice (e.g., utilizing an anthropomorphic phantom that mimics human vasculature to perform method). In various embodiments, the methods can be performed on a cadaver heart, simulator (e.g. with the body parts, tissue, etc. being simulated), anthropomorphic phantom, etc.

When a transcatheter delivery system is used, any appropriate approach may be utilized to reach the site of deployment, including (but not limited to) a transfemoral, subclavian, transapical, or transaortic approach. In several embodiments, a catheter containing an expandable device is delivered in a crimped state via a guidewire to the site of deployment. At the site of deployment, in accordance with many embodiments, an expandable device is released from the catheter and then expanded into form such that the central core has a controlled internal diameter and the appendages expand outward to the luminal wall. A number of expansion mechanisms can be utilized, such as (for example) an inflatable balloon, mechanical expansion, or utilization of a self-expanding mechanism. In some embodiments, the central core of an expandable device has a higher resistance to expansion, which may help the central core reach, but not expand beyond, the controlled internal diameter. Particular shape designs and radiopaque regions on the frame and/or on the cover can be utilized to monitor the expansion and implementation.

Once an expandable device is expanded, in accordance with several embodiments, the distal appendages of the expandable device are engaged with the luminal wall via radial forces and/or attachment. In various embodiments utilizing an impermeable cover, sealing portions are engaged with the luminal wall via radial forces and/or attachment. In various embodiments utilizing a prosthetic device, the device can be delivered to the site of deployment by a catheter, released, and expanded within the central core of the docking station such that it engages inner core (e.g., valve seat) via radial forces and/or attachment.

Delivery and employment of an expandable device may be utilized in a variety of applications. In some embodiments, an expandable device is delivered to a site experiencing an aneurysm, especially an aortic aneurysm. In various embodiments, an expandable device, when utilized with an impermeable cover, can be used as a graft and guide blood flow to bypass an aneurytic site, which may help prevent dissection and/or bursting of a vascular wall. In some embodiments, an expandable device with an impermeable cloth can be utilized as an aortic graft for individuals with a dilated ascending aorta. In some embodiments, an expandable device can be utilized to deploy a prosthetic valve, especially in sites that are dilated.

Docking Station with Bent Arm

Various embodiments of docking station devices having a bent arm are described. In several embodiments, a docking device with a bent arm is used along with a prosthetic valve to supplement the function of a defective tricuspid valve and/or to prevent too much pressure from building up in the right atrium (RA) (see FIG. 1). During systole, the leaflets of a normally functioning tricuspid valve (TV) close to prevent the venous blood from regurgitating back into the right atrium (RA). When the tricuspid valve does not operate normally, blood can backflow or regurgitate into the right atrium (RA), the inferior vena cava (IVC), the superior vena cava (SVC), and/or other vessels in the systolic phase. Blood regurgitating backward into the right atrium increases the volume of blood in the atrium and the blood vessels that direct blood to the heart. This can cause the right atrium to enlarge and cause blood pressure to increase in the right atrium and blood vessels, which can cause damage to and/or swelling of the liver, kidneys, legs, other organs, etc. A transcatheter valve or THV implanted in the inferior vena cava (IVC) and/or the superior vena cava (SVC) can prevent or inhibit blood from backflowing into the inferior vena cava (IVC) and/or the superior vena cava (SVC) during the systolic phase.

In accordance with several embodiments, a docking station with a bent arm is capable of adapting to and situating within the IVC and/or SVC at the interface with the RA. In many embodiments, a docking station with a bent arm has an expandable cylindrical frame with an inflow section at one distal end, an outflow section at the other distal end and central portion therebetween. In numerous embodiments, when expanded, the outflow section has an arm that extends outward and is bent to form an elbow-like conformation, which can be utilized to engage onto a portion of the inner RA wall when implanted. In multiple embodiments, an outflow section having a bent arm is generally open, which should allow to blood to flow freely. In accordance with many embodiments, a docking station with a bent arm can expand radially outward from an unexpanded form such that the inflow portion can expand to a luminal wall of the IVC (above the hepatic veins) or SVC, and expansion can further release and situate the bent arm into place. When expanded, in many embodiments, a docking station fits to the SVC/RA or IVC/RA interface such that inflow section is within the SVC or IVC and the outflow section reaches into the RA via a bent arm that is capable of engaging with the inner RA wall.

In several embodiments, a docking station with a bent arm is formed of a number interconnecting struts, including the bent arm, to form a number of cells. In numerous embodiments, each cell within the docking station is connected and/or adjacent to another cell to form rows of cells, each row of cells circulating around the circumference of the docking station. In some embodiments, the outflow section only forms cells partially around the circumference of the docking station, leaving a gap where a bent arm can be situated. Having a gap at within the outflow section where the bent arm is located could enhance blood flow into the RA when situated. In regard to cell size and shape, it should be understood that the perimeter of a cell can vary, depending on the length of the struts that form the cell. It should be further understood that the width or thickness of a strut can vary, which may be beneficial to provide rigidity or malleability along some portions of the strut.

In many embodiments, the bent arm of the docking station is an elongated cell that extends upward from the middle section of the docking station and bends outward. The size and shape of the bent arm can vary, as long it is capable of engaging the SVC/RA or IVC/RA interface. It is important to consider the length and the location of the bend such that it bends at the SVC/RA or IVC/RA interface, but is not so long that it interferes with tricuspid valve function.

In a number of embodiments, the cells of a docking station are capable of constricting into an unexpanded form and/or crimped state, typically resulting in an elongated length and shortened circumference. In many embodiments, the bent arm is straightened and elongated when the docking station is in unexpanded form. In various embodiments, when a docking is expanded outwardly from an unexpanded form, the length is shortened, the circumference of the device is increased, and the arm bends into place. Accordingly, in several embodiments, a docking station with a bent arm can be expanded at the site of deployment, such that the inflow section is expanded to reach outward to the luminal walls of SVC or IVC (above the hepatic veins) and the bent arm engages SVC/RA or IVC/RA interface. In many embodiments, a central section has an expanded circumference that is restricted such that the internal diameter of the central section is controlled regardless of the SVC or IVC lumen size.

In several embodiments, the central section of a docking device is less expandable, such that the central section is narrower than the inflow or outflow sections when expanded. In numerous embodiments, the narrow central section is utilized as a valve seat, which can situate a valve therein. In some embodiments, the inner diameter of the central section is compatible with the outer diameter of a prosthetic valve. Having a controlled inner diameter can help ensure that a manufactured valve having a standard diameter will work in any local vascular environment, despite the size, contour and dimensionality of the SVC or IVC.

In many embodiments, the internal diameter of the central section is between 15 mm and 35 mm. In various embodiments, an internal diameter is approximately one of: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm. It is to be understood, however, that the controlled inner diameter can be any appropriate size to house a prosthetic.

In various embodiments, the diameter of the inflow section and/or the diameter of the outflow section is larger than the diameter of a central section, conferring an “hour glass” shape. In other words, in some embodiments, the inflow and/or outflow sections are capable of expanding in an outward direction beyond the circumference of the central section, which may be useful to reach luminal walls of the SVC or IVC.

In many embodiments, the expanded inflow section and/or the expanded outflow section, along with the bent arm, help secure an implanted device at the SVC/RA or IVC/RA interface, and mitigate sliding and/or dislodging from the implantation site. In several embodiments, to help secure location, the expanded inflow section and/or the expanded outflow section provide a radial force in the contact with the luminal wall. In many embodiments, the bent arm engages with the wall of the RA prevents the docking station from sliding away from the SVC/RA or IVC/RA interface. Preventing sliding within the IVC can help prevent the docking station from covering the interconnections between the hepatic veins and IVC.

In some embodiments, struts of a docking may be shaped and/or have radiopaque qualities at various points along the struts, such that the docking station orientation and placement can be easily recognized utilizing appropriate radiographic imaging technique (e.g., sonography). Facilitating visualization of the docking can be useful to ensure that the inflow and outflow sections appropriately engage the luminal wall and that the bent arm appropriately engages the RA wall. In some embodiments, shapes and/or radiopaque portions are integrated to help identify the bent arm. For example, in some embodiments, a particular shape and/or radiopaque portion can be integrated onto the docking station directly below and/or opposite to the bent arm.

In several embodiments, a docking station with a bent arm is composed of a resilient and compliant material capable of expanding at the site of deployment. In some embodiments, a docking station is made of a self-expanding material such as (for example) nitinol. A self-expanding material may continue to expand within the site of deployment, helping it to secure within its place by radial expansion forces. In some embodiments, a docking station is made of cobalt chromium or stainless steel.

In a number of embodiments, a skirt cover is attached to the docking station with a bent arm such that a wall is formed around the outer circumference and along the length of the docking station and a lumen is formed within the cover, having an opening at both ends of the expandable device. In many embodiments, a cover is attached at the inflow end. In some embodiments, a cover is also attached along the circumference of the central section. In some embodiments, a cover is only attached to a portion of the docking station, such that a wall is formed for only a portion along the length of the docking station. In some embodiments, a partial cover covers the inflow end and/or outflow end and/or central core. In some embodiments, a partial cover covers the inflow end and central core, leaving the outflow end relatively open. In some embodiments, a cover is attached to the inner wall of the docking station. In some embodiments, a cover is attached to the outer wall of the docking station. In some embodiments, a cover is attached to both the inner wall and outer wall of the docking station.

In some embodiments, a cover can include radiopaque portions. In some embodiments, radiopaque portions are integrated within a cover. In some embodiments, radiopaque portions are stitched onto and/or within a cover. In some embodiments, radiopaque portions are bonded onto and/or within a cover.

In many embodiments, a cover is impermeable to blood and constituents of the circulatory system. In several embodiments, a cover is a biocompatible fabric (e.g., PET cloth) or a bioprosthetic tissue. Examples of bioprosthetic tissue include (but are not limited to) animal pericardium and small intestinal submucosa (SIS). Bioprosthetic tissue can be derived from any appropriate animal source, including (but not limited to) bovine, porcine, ovine, avian, and human donor.

In various embodiments utilizing an impermeable cover, sealing portions of a cover can be utilized to seal a docking station to a luminal wall, ensuring blood flows through the docking station and not around it. In several embodiments, a sealing portion is integrally formed onto the body of the docking station with a cover, in which a portion of the cover makes contact with the inner wall when expanded at the site of deployment. In many embodiments, a sealing portion is utilized on the inflow end of a device. In some embodiments, a sealing portion is utilized on the outflow end of a device. Utilizing a cover with sealing portions on a docking can be beneficial to ensure blood flows through the central section and a prosthetic valve, which can mitigate backflow from the RA back into the SVC or IVC.

In several embodiments, a docking station is utilized to situate a prosthetic valve at the at the SVC/RA or IVC/RA interface. In many embodiments, a prosthetic valve is utilized to help control the flow of blood and mitigate backflow. In multiple embodiments, the central section of the docking station is utilized as a valve seat. In some embodiments, the inner diameter of a docking station's central section is compatible with the outer diameter of the prosthetic valve. Having a controlled inner diameter can help ensure that a manufactured valve having a standard diameter will work in any local vascular environment, despite the size, contour and dimensionality of the local environment.

In many embodiments, a prosthetic device has an outer diameter, the outer diameter being a size between 15 mm and 35 mm. In various embodiments, an outer diameter is approximately one of: 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or 35 mm. It is to be understood, however, that the prosthetic device outer diameter can be any appropriate size and a docking station inner diameter can be utilized to be compatible the outer diameter of the prosthetic device.

In some embodiments, a prosthetic device is self-expanding, utilizing radial forces to secure itself within the central section of a docking station. In some embodiments, a mechanism is utilized to secure a prosthetic device with the central core of a docking station, such as (for example) a latch or hook.

A number of prosthetic valves can be utilized with a docking station, including (but not limited to) mechanical and tissue-based valves. A number of valves have been described and can be utilized, including valves described in U.S. Pat. No. 8,002,825, Published Patent Cooperation Treaty Application No. WO 2000/42950, U.S. Pat. Nos. 5,928,281, 6,558,418, 6,540,782, 3,365,728, 3,824,629, and 5,814,099, the disclosure of which are each incorporated herein by reference in each patent's or publication's entirety. In some embodiments, the Edwards Lifescience's SAPIEN Transcatheter Heart Valve is utilized. It is noted that any appropriate valve capable of fitting within an expandable device, as described herein, can be utilized in accordance with various embodiments of the invention.

In many embodiments, a docking station with bent arm can be inserted within a transcatheter delivery device. Accordingly, a docking station in an unexpanded form and crimped state is inserted within a catheter such that the docking station can be delivered via the transcatheter approach to a site of implementation. Any appropriate transcatheter delivery system can be employed. In some embodiments, a docking station utilizes a transcatheter delivery system described in the US Patent Publication No. 2017/0231756, the disclosure of which is incorporated herein by reference it its entirety.

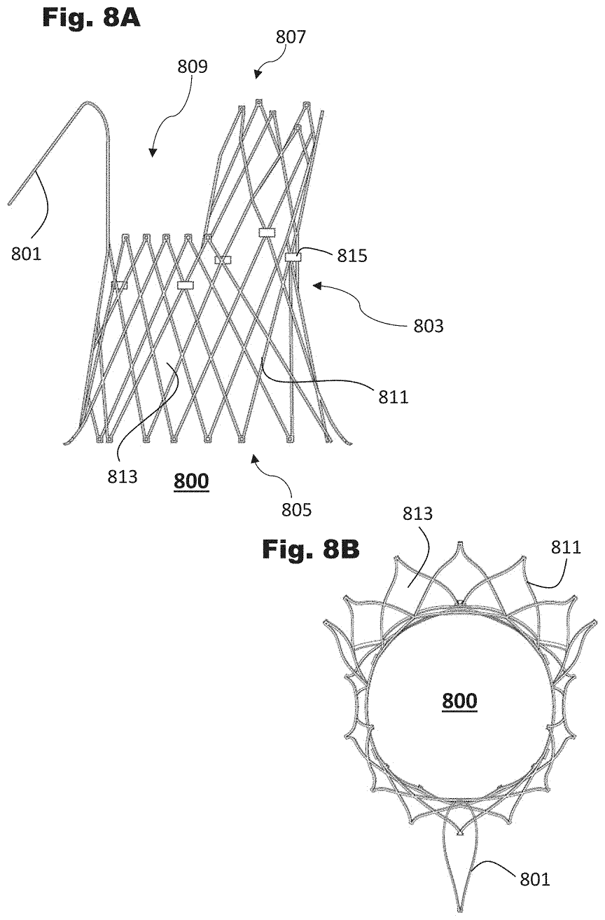

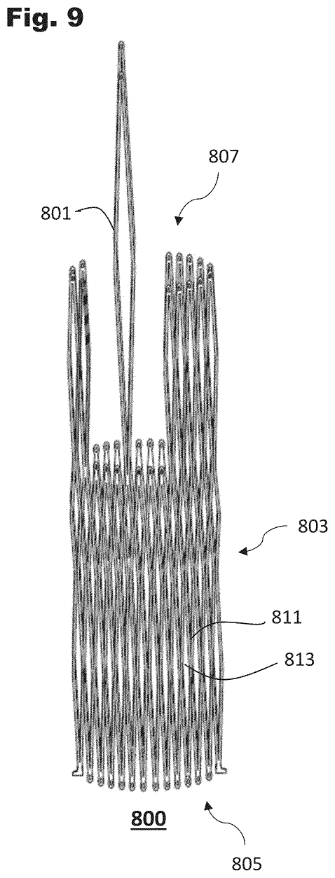

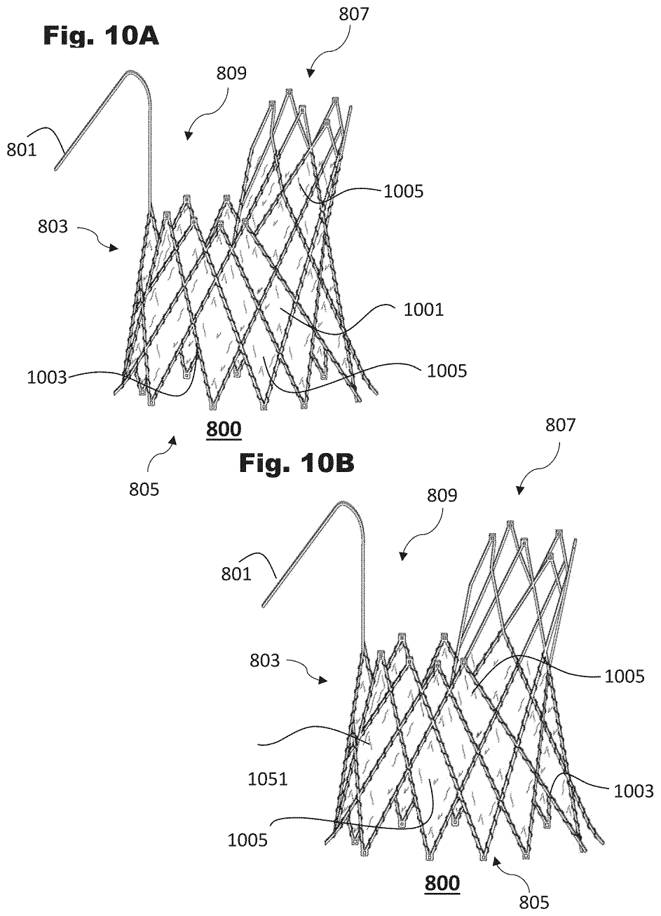

Provided in FIGS. 8A and 8B are a side view and top-down view of a docking station 800 having a bent arm 801 shown in expanded form. As shown, the docking station 800 has a cylindrical/hour glass shape with a central section 803, an inflow end 805, and an outflow end 807. The inflow and outflow ends 805 and 807 expand further outward than central section 803, allowing the inflow and outflow ends to contact and engage with an inner wall at the site of deployment. The outflow end 807 includes a gap 809 and the bent arm 801 within the gap. Docking station 800 is formed by a number of interconnecting struts 811 forming a number of cells 813 that allow the docking station to expand and constrict. As shown, the docking station 800 includes a number of radiopaque shapes 815 included as a part of the frame, which can facilitate visualization of the docking station during implantation at the target site.

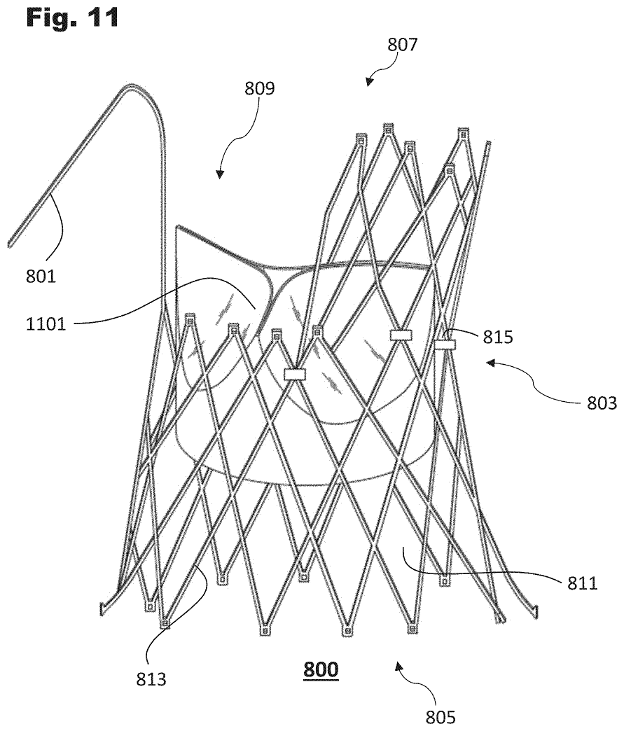

FIG. 9 portrays a docking station 800 in an unexpanded configuration. As shown the cylindrical diameter is reduced while the length of the device is expanded. The expandable device 300 in a crimped state can be inserted within a catheter such that it can be delivered by a transcatheter approach. As shown, the arm 801 is straightened out and elongated such that it can fit within a catheter. It is noted the arm 801 can be condensed into any appropriate conformation that allows the docking station 800 to fit within a catheter.

FIGS. 10A and 10B portray docking station 800 with a cover attached thereupon. A full length cover 1001 that extends from inflow end 805 to the outflow end 807 is shown in FIG. 10A. Alternatively a partial length cover 1051 that extends from inflow end 805 to just beyond the central section 803 and up to the gap 809 of the outflow section 807 is shown in FIG. 10B. The full length cover 1001 and partial length cover 1051 can be attached to the struts 811 of the frame via stitching 1003 or any other appropriate means. The covers 1001 and 1051 can be made of an appropriate material, such as (for example) a biocompatible fabric (e.g., PET cloth) or a bioprosthetic tissue (e.g., animal pericardium or small intestinal submucosa). Furthermore, the covers 1001 and 1051 can be made impermeable to blood and its constituents such that it can guide the blood flow throw the central section 803.

Sealing portions 1005 are formed on the covers 1001 and 1051 towards the inflow and outflow ends 803 and 805. The sealing portions 1005 can engage with an inner wall at the site of deployment. Having sealing portions 1005 engaged with a luminal wall forms a seal that ensures that blood flows through the central section 803 and does not bypass.

Provided in FIG. 11 is docking station 800 supporting a prosthetic valve 1101. The valve 1101 is situated within the central section 803. The inner circumference of the central section 803 can be controlled such that it is compatible with the outer circumference of the prosthetic valve 1101. The prosthetic valve 1101 can be self-expanding and utilize radial forces to secure itself within the central core 803 or a mechanism can be utilized to secure the prosthetic valve 1101, such as (for example) a latch or hook. Any appropriate prosthetic valve 1101 may be utilized, including (but not limited to) mechanical and tissue-based valves. It is noted that the prosthetic valve 1101 can be incorporated within docking station 800 having a cover 1001 or 1051, which would help guide the blood flow through the prosthetic valve.

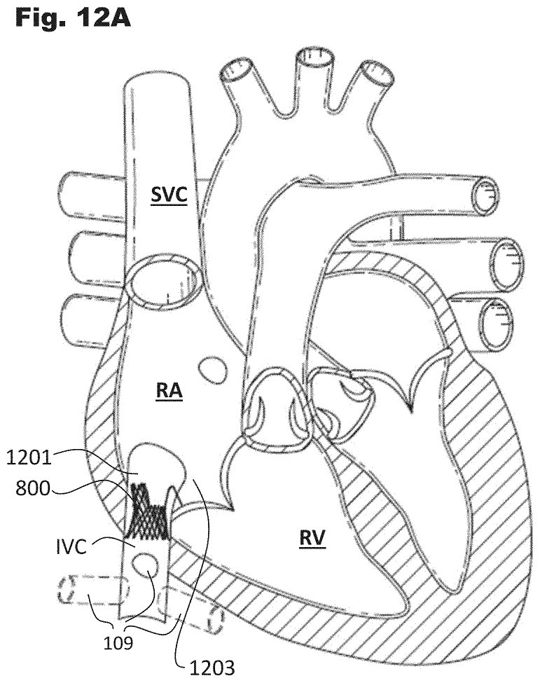

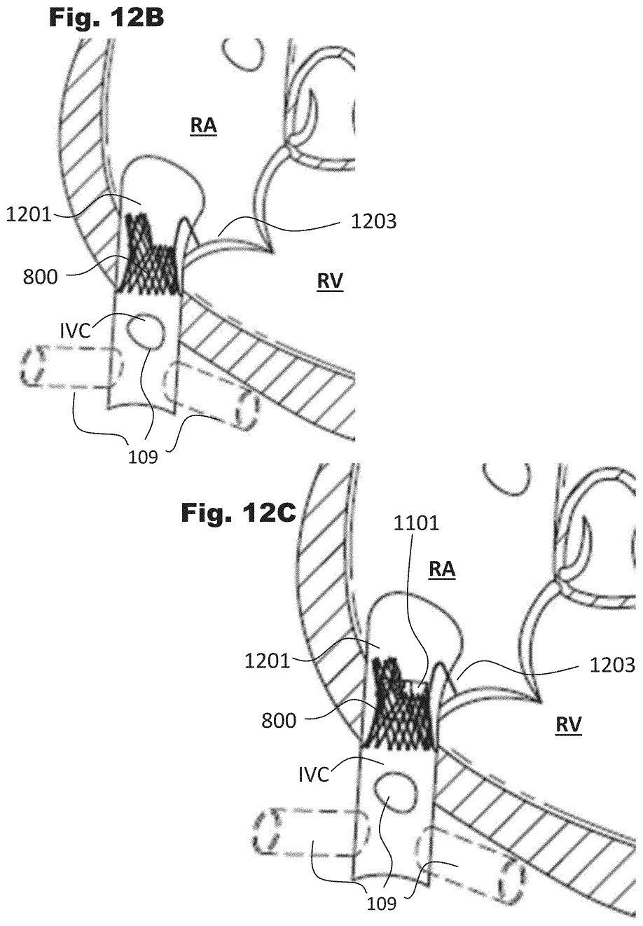

FIGS. 12A to 12C portray docking station 800 inserted within the interconnection of the SVC and RA. When inserted at the site of deployment, the inflow end 803 expands outward and engages with the luminal wall 1201 of the SVC. The outflow end 805 opposite of the gap 809 can engage with the luminal wall while the bent arm 801 can insert itself into the RA and engage with the wall 1203 of the RA. The inflow and outflow ends 803 and 805 and the bent arm 801 can be secured to luminal wall utilizing radial force and/or an attachment mechanism. Attachment mechanisms include (but are not limited to) utilization of orifices and sutures or hooks. The bent arm 801 can help mitigate docking station 800 from flowing back in to the SVC and covering the hepatic veins. FIG. 12C shows docking station 800 incorporating a prosthetic valve 1101. Although not shown in FIGS. 12A to 12C, a cover 1001 or 1051 can also be utilized. It should be understood that docking station 800 could also be used within the SVC and RA interconnection.

A number of embodiments are directed to methods of delivering a docking station with bent arm to the site of deployment. A method can be performed on any suitable recipient, including (but not limited to) humans, other mammals (e.g., porcine), cadavers, or anthropomorphic phantoms, as would be understood in the art. Accordingly, methods of delivery include both methods of treatment (e.g., treatment of human subjects) and methods of training and/or practice (e.g., utilizing an anthropomorphic phantom that mimics human vasculature to perform method). In various embodiments, the methods can be performed on a cadaver heart, simulator (e.g. with the body parts, tissue, etc. being simulated), anthropomorphic phantom, etc.

When a transcatheter delivery system is used, any appropriate approach may be utilized to reach the site of deployment, including (but not limited to) a transfemoral, subclavian, transapical, or transaortic approach. In several embodiments, a catheter containing a docking station is delivered via a guidewire to the site of deployment. At the site of deployment, in accordance with many embodiments, a docking station is released from the catheter and then expanded into form such that the outflow end expands outward to the luminal wall and the bent arm engages the inner wall of the RA, followed by the central portion and the inflow end. A number of expansion mechanisms can be utilized, such as (for example) an inflatable balloon, mechanical expansion, or utilization of a self-expanding device. In some embodiments, the central section of a docking station does not expand as far as the distal ends, such that it provides an appropriate internal diameter to situate a prosthetic valve. Particular shape designs and radiopaque regions on the frame can be utilized to monitor the expansion and implementation.

Once a docking station is expanded, in accordance with several embodiments, the distal ends of the docking station are engaged with the luminal wall via radial forces and/or attachment. In various embodiments utilizing an impermeable cover, sealing portions are engaged with the luminal wall via radial forces and/or attachment. In various embodiments utilizing a prosthetic device, the device can be delivered to the site of deployment by a catheter, released, and expanded within the central section of the docking station such that it engages inner core (e.g., valve seat) via radial forces and/or attachment.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. An expandable device for implanting within vasculature, comprising:
a central core comprising a number of struts that form a number of cells, the cells forming a circumference encircling the central core;
wherein the central core is capable of expanding from an unexpanded state radially outward to an expanded state;
wherein when the central core is an expanded state, the central core has a controlled inner diameter; and
a plurality of appendages that extend away from the central core, each appendage is formed by at least one strut and is independent from the other appendages;
wherein each appendage is capable of independently expanding from an unexpanded state radially outward to an expanded state such that an apex of each appendage extends radially outward beyond an outer circumference of the central core.

2. The expandable device of claim 1 wherein the controlled inner diameter of the central core in the expanded state is between 15 mm and 35 mm.

3. The expandable device of claim 1 wherein at least one appendage is formed by at least two struts that connect at the apex of the appendage.

4. The expandable device of claim 1 wherein at least one appendage forms a V-shape.

5. The expandable device of claim 1 wherein at least one appendage forms a Y-shape.

6. The expandable device of claim 1 wherein at least one appendage has an orifice at the apex.

7. The expandable device of claim 1 wherein at least one appendage has a hook at the apex.

8. The expandable device of claim 1 further comprising a radiopaque portion within the central core or an appendage.

9. The expandable device of claim 1 wherein a cover is incorporated within the central core.

10. The expandable device of claim 9 wherein the cover is secured to the struts of the central core.

11. The expandable device of claim 9 wherein the cover is a biocompatible fabric.

12. The expandable device of claim 9 wherein the cover is a bioprosthetic tissue.

13. The expandable device of claim 9 wherein the cover is further incorporated within the plurality of appendages and includes sealing portions that are capable of engaging an inner wall of vasculature at a site of deployment.

14. The expandable device of claim 1 wherein the central core is configured to engage a prosthesis.

15. The expandable device of claim 1 wherein the central core includes a valve seat configured to situate a prosthetic valve.

16. The expandable device of claim 1 wherein the central core includes a valve seat configured to situate a prosthetic valve having an outer diameter between 15 mm and 35 mm.

17. The expandable device of claim 1 wherein, when the central core and the plurality of appendages are in a crimped state, the central core and the plurality of appendages are configured to be inserted within a catheter.

18. The expandable device of claim 1 wherein, when in the expanded state, the apex of each appendage is capable of engaging an inner wall of a vasculature.

19. An expandable device for implanting within vasculature to treat an aneurysm, the device comprising:
a central core comprising a number of struts that form a number of cells, the cells forming a circumference encircling the central core;
wherein the central core is capable of expanding from an unexpanded state radially outward to an expanded state;
wherein when the central core is an expanded state, the central core has an inner diameter;
a plurality of appendages that extend away from the central core, each appendage is formed by at least one strut and is independent from the other appendages; and
a cover secured to the struts of the central core, the cover including sealing portions that are capable of engaging an inner wall of vasculature at a site of deployment;
wherein each appendage is capable of expanding from an unexpanded state radially outward to an expanded state such that an apex of each appendage extends radially outward beyond an outer circumference of the central core.

20. The expandable device of claim 19 wherein at least one appendage is formed by at least two struts that connect at the apex of the appendage.

21. The expandable device of claim 19 wherein at least one appendage forms a V-shape.

22. The expandable device of claim 19 wherein at least one appendage forms a Y-shape.

23. The expandable device of claim 19 wherein at least one appendage has an orifice at the apex.

24. The expandable device of claim 19 wherein at least one appendage has a hook at the apex.
